(19) United States
(12) Patent Application Publication
(10) Pub. No.: US 2018/0264093 A1
(43) Pub. Date: Sep. 20, 2018

(54) IMMUNOGENIC GLYCOPEPTIDE COMPOUNDS, PHARMACEUTICAL COMPOSITIONS AND USES THEREOF

(71) Applicants: MACKAY MEMORIAL HOSPITAL OF TAIWAN PRESBYTERIAN CHURCH AND MACKAY MEMORIAL SOCIAL WORK FOUNDATION, Taipei City (TW); DCB-USA, LLC, Wilmington, DE (US)

(72) Inventors: Chih-Long CHANG, Taipei City (TW); Chao-Chih WU, Taipei City (TW)

(21) Appl. No.: 15/761,301

(22) PCT Filed: Sep. 14, 2016
(86) PCT No.: PCT/US16/51702

§ 371 Date: Mar. 19, 2018

Related U.S. Application Data
(60) Provisional application No. 62/220,923, filed on Sep. 18, 2015.

(51) Int. Cl. A61K 39/00; A61K 39/395; A61P 35/00

(52) U.S. Cl. CPC A61K 39/0011; A61K 39/39558; A61P 35/00; A61K 2039/70; A61K 2039/55516; A61K 2039/55583; A61K 2039/55505

(57) ABSTRACT

Disclosed herein are immunogenic glycopeptide compounds for inducing immune responses to prevent and/or treat cancer. Other aspects of the present disclosure are pharmaceutical compositions comprising the immunogenic glycopeptide compounds, and methods using the compounds for preventing and/or treating a cancer in a subject.

### FIELD

The present invention relates to the field of immunotherapy of cancer. In particular, the disclosed invention relates to an immunogenic glycopeptide, a pharmaceutical composition comprising the glycopeptide and to the use thereof for enhancing the immune response and notably in cancer therapy.

### REFERENCE TO SEQUENCE LISTING

The official copy of the Sequence Listing is submitted concurrently with the specification as an ASCII formatted text file via EFS-Web, with a file name of “05384P002PV_SeqListing.txt”, a creation date of Sep. 18, 2015, and a size of 1062 bytes. The Sequence Listing filed via EFS-Web is part of the specification and is incorporated in its entirety by reference herein.

### BACKGROUND

Globo H (Fucα1→2Galβ1→3GalNAcβ1→3Galα1→4Galβ1→4Glcβ1→O-cer) is a hexasaccharide and belongs to a large number of tumor-associated carbohydrate antigens that are overexpressed on the surface of various epithelial cancer cells, including breast, colon, ovarian, pancreatic, lung, and prostate cancer cells. The aberrant expression of Globo H renders it an attractive candidate for immunotherapy and the development of cancer vaccines for Globo H-expressing cancers. In addition to Globo H, other known carbohydrate antigens including GM2, GD2, GD3, fucosyl-GM1, Lewis^{y} (Le^{y}, Fucα1→2Galβ1→4[Fucα1→3]GlcNAcβ1→3Galβ1→O-cer), Tn (GalNAcα-O-Ser/Thr), TF (Galβ1→3GalNAcα-O-Ser/Thr) and STn (NeuAcα2→6GalNAcα-O-Ser/Thr) are also used as target antigens for cancer immunotherapy (Susan F Slovin et al., Carbohydrate Vaccines as Immunotherapy for Cancer, Immunology and Cell Biology (2005) 83, 418-428; Zhongwu Guo and Qianli Wang, Recent Development in Carbohydrate-Based Cancer Vaccines, Curr Opin Chem Biol. 2009 December; 13(5-6): 608-617; Therese Buskas et al., Immunotherapy for Cancer: Synthetic Carbohydrate-based Vaccines, Chem Commun (Camb). 2009 Sep. 28; (36): 5335-5349).

However, most carbohydrate antigens are often tolerated by the immune system, and consequently, the immunogenicity induced by them is limited. Further, the production of antibody against a specific immunogen typically involves the cooperative interaction of two types of lymphocytes, B-cells and helper T-cells. For example, Globo H alone cannot activate helper T-cells, which also attributes to the poor immunogenicity of Globo H. Accordingly, the immunization with Globo H is often typified by a low titer of immunoglobulin M (IgM) and a failure to class switch to immunoglobulin G (IgG), as well as ineffective antibody affinity maturation.

Various approaches have been developed to address the above-mentioned deficiencies. In certain researches, foreign carrier proteins or peptides having T-epitopes (such as keyhole limpet hemocyanin (KLH) or detoxified tetanus toxoid (TT)) have been conjugated with carbohydrate antigens hoping to enhance the immunogenicity of the carbohydrate antigens. US 20010048929 provided a multivalent immunogenic molecule, comprising a carrier molecule containing at least one functional T-cell epitope, and multiple different carbohydrate fragments each linked to the carrier molecule and each containing at least one functional B-cell epitope, wherein said carrier molecule imparts enhanced immunogenicity to said multiple carbohydrate fragments and wherein the carbohydrate fragment is Globo H, Le_{Y} or STn. US 20120328646 provides a carbohydrate based vaccine containing Globo H (B cell epitope) chemically conjugated to the immunogenic carrier diphtheria toxin cross-reacting material 197 (DT-CRM 197) (Th epitope) via a p-nitrophenyl linker, which provides immunogenicity in breast cancer models, showing delayed tumorigenesis in xenograft studies. US 20120263749 relates to a polyvalent vaccine for treating cancer comprising at least two conjugated antigens selected from a group containing glycolipid antigen such as Globo H, a Lewis antigen and a ganglioside, polysaccharide antigen, mucin antigen, glycosylated mucin antigen and an appropriate adjuvant.

Furthermore, conjugation of carbohydrates to a carrier protein poses several new problems. According to Ingale et al., the foreign carrier protein and the linker for attaching the carrier protein and the carbohydrate may elicit strong B-cell responses, thereby leading to the suppression of an antibody response against the carbohydrate epitope (Ingale S. et al., “Robust immune responses elicited by a fully synthetic three-component vaccine,” Nat Chem Biol. 2007 October; 3(10):663-7. Epub 2007 Sep. 2). For example, Buskas et al., taught that conjugation of carbohydrates using a Huisgen cycloaddition “click reaction” introduces a rigid triazole moiety, which may be immunogenic and further suppress the low immunogenicity of tumor-associated carbohydrate antigens (Buskas et al., “Immunotherapy for Cancer: Synthetic Carbohydrate-based Vaccines,” Chem. Commun. (Camb). 2009 Sep. 28; (36): 5335-5349. Doi: 10.1039/b908664c). Ingale et al. also teaches that the conjugation chemistry is difficult to control, resulting in conjugates with ambiguities in composition and structure, which may affect the reproducibility of an immune response.

Considering the above-mentioned difficulties associated with conjugating carbohydrates and proteins while maintaining proper immunogenicity, Ingale et al. concluded that it is not surprising that preclinical and clinical studies using carbohydrate-protein conjugates have led to mixed results. For example, Kuduk et al. taught that the immunization with a trimeric cluster of Tn-antigens conjugated to KLH in the presence of the adjuvant QS-21 elicited only modest titers of IgG antibodies in mice (Kuduk S D, et al. “Synthetic and immunological studies on clustered modes of mucin-related Tn and TF O-linked antigens: the preparation of a glycopeptide-based vaccine for clinical trials against prostate cancer,” J Am Chem Soc. 1998; 120:12474-12485). Slovin et al. taught that the same vaccine gave low median IgG and IgM antibody titers in a clinical trial of relapsed prostate cancer patients (Slovin S F, et al., “Fully synthetic carbohydrate-based vaccines in biochemically relapsed prostate cancer: clinical trial results with alpha-N-acetylgalactosamine-O-serine/threonine conjugate vaccine,” J Clin Oncol. 2003; 21:4292-4298).

Moreover, for cancer patients with hypoimmune status, particularly patients receiving chemotherapy or radiation therapy, and late-stage cancer patients, the efficacy of active immune intervention is often limited, for these patients may not be able to produce sufficient antibodies to elicit the anti-tumor effect.

In view of the foregoing, there remains a strong need in the art for developing alternative strategies for improving the immunogenicity and/or therapeutic efficacy of carbohydrate-based immunogens for use as vaccines and/or production of therapeutic antibodies.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to immunogenic glycopeptide compounds or derivatives thereof, wherein the immunogenic glycopeptide compounds or derivatives thereof can elicit high titers of immunoglobulin G (IgG) and immunoglobulin M (IgM) antibodies in vivo against carbohydrate antigens selected from Globo H, SSEA4, GD2, GD3, GM2, fucosyl GM1, LewisY Le(y), sialyl-Le(x), sialyl-Le(a), TF, Tn and sialyl-Tn. In one aspect, the immunogenic glycopeptide compounds or derivatives thereof can elicit much higher titers of IgG antibodies relative to IgM antibodies, a characteristic particularly advantageous for the development of highly specific therapeutic antibodies, such a chimeric or humanized antibodies against tumor associated carbohydrate antigens that can be used in the treatment of cancers.

In one aspect the immunogenic glycopeptide compound of the present disclosure has structural formula (I)

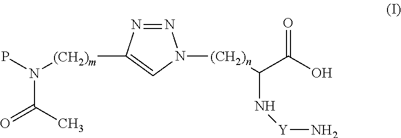

wherein P is a carbohydrate antigen selected from Globo H, SSEA4, GD2, GD3, GM2, fucosyl GM1, LewisY Le(y), sialyl-Le(x), sialyl-Le(a), TF, Tn and sialyl-Tn; m=1 to 4; Y is a pan-DR epitope comprising an amino acid sequence at least 80% identical to AKXVAAWTLKAAA (SEQ ID NO: 1), wherein X is an amino acid residue selected from cyclohexylalanine, phenylalanine, and tyrosine; and n=1 to 5.

In some aspects, the immunogenic glycopeptide compound of structural formula (I), the compound structure can have m=1, and/or n=4.

In some aspects, the immunogenic glycopeptide compound of structural formula (I), the pan-DR epitope consists of the amino acid sequence AKXVAAWTLKAAA (SEQ ID NO: 1) or the amino acid sequence AKXVAAWTLKAA (SEQ ID NO: 2).

In some aspects of the immunogenic glycopeptide compound of structural formula (I), X is a cyclohexylalanine.

In some aspects, the immunogenic glycopeptide compound of structural formula (I), the carbohydrate antigen is Globo H.

In some aspects, the immunogenic glycopeptide compound has structural formula (II)

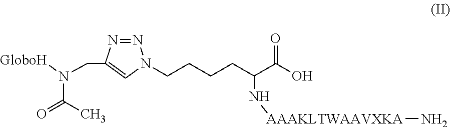

wherein, “GloboH” is the carbohydrate antigen, Globo H, and X is cyclohexylalanine.

In some aspects, the immunogenic glycopeptide compound has structural formula (III)

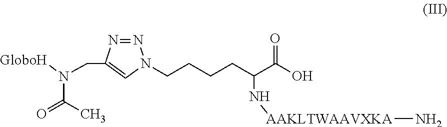

wherein, “GloboH” is the carbohydrate antigen, Globo H, and X is cyclohexylalanine.

In another aspect, the present disclosure also provides pharmaceutical compositions comprising the immunogenic glycopeptide compounds of structural formulae (I), (II), (III), and others disclosed herein. Accordingly, in another embodiment the present disclosure provides a pharmaceutical composition comprising a therapeutically effective amount of an immunogenic glycopeptide compound as disclosed herein (e.g., compound of structural formulae (I), (II), or (III)), and a pharmaceutically acceptable carrier or adjuvant. In some aspects, the adjuvant is QS21 or aluminum hydroxide.

In some aspects of the pharmaceutical composition embodiments disclosed herein, the composition is a vaccine. In some embodiments, the vaccine is a polyvalent vaccine comprising two or more immunogenic glycopeptide compounds as disclosed herein (e.g., compound of structural formula (I)), and each of the of the two or more compounds has a different carbohydrate antigen selected from Globo H, SSEA4, GD2, GD3, GM2, fucosyl GM1, LewisY Le(y), sialyl-Le(x), sialyl-Le(a), TF, Tn and sialyl-Tn. In some embodiments of the polyvalent vaccine composition, the two or more compounds comprise the carbohydrate antigens: Globo H, SSEA4, GD2, GD3, GM2, fucosyl GM1, LewisY Le(y), sialyl-Le(x), sialyl-Le(a), TF, Tn and sialyl-Tn.

In another aspect of the various embodiments of immunogenic glycopeptide compounds and pharmaceutical compositions disclosed herein, the compound or composition has the characteristic of eliciting increased production of IgG relative to IgM antibodies in mice immunized with them. Thus, in some embodiments, the disclosure provides immunogenic glycopeptide compounds and/or pharmaceutical compositions comprising said compounds, wherein mice immunized with the compound or composition produce a higher titer of IgG relative to IgM antibodies specific to the carbohydrate antigen. In some embodiments of the compounds and compositions, the titer of IgG relative to IgM antibodies specific to the carbohydrate antigen produced in immunized mice is increased at least about 2-fold, at least about 4-fold, at least about 5-fold, or at least about 10-fold.

In another aspect, the present disclosure provides methods for preventing and/or treating a cancer in a subject comprising administering to the subject an effective amount of an immunogenic glycopeptide compound as disclosed herein (e.g., compound of structural formulae (I), (II), or (III)).

In one aspect of the methods for preventing and/or treating a cancer in a subject disclosed herein, the method comprises administering to the subject an effective amount of the pharmaceutical composition as disclosed herein, e.g., a therapeutically effective amount of an immunogenic glycopeptide compound as disclosed herein and a pharmaceutically acceptable carrier or adjuvant.

In one aspect of the methods for preventing and/or treating a cancer in a subject, the cancer is a tumor-associated carbohydrate-expressing cancer. In one aspect of the methods, the cancer is breast cancer, ovarian cancer, pancreatic cancer, prostate cancer, colorectal cancer or lung cancer.

Many of the attendant features and advantages of the present disclosure will becomes better understood with reference to the following detailed description considered in connection with the accompanying drawings.

### BRIEF DESCRIPTION OF THE DRAWINGS

FIGS. 1A to E illustrate results of cell binding assay (A: Isotype; B: VK9; C: MZ-2; D: Control serum; E: α-Globo H serum).

FIGS. 2(A) and (B) provide bar graphs illustrating the IgM titers of mice immunized with the MZ-11-Globo H glycopeptide compound prepared as in Example 1 (i.e., compound of formula (II), wherein X is cyclohexylalanine). FIG. 2(A) shows results for diluted serum IgM 1:100 and FIG. 2(B) for diluted serum IgM 1:1000.

FIGS. 3(A) and (B) provide bar graphs illustrating the IgG titers of mice immunized with the MZ-11-Globo H glycopeptide compound prepared as in Example 1. FIG. 3(A) shows results for diluted serum IgG 1:100 and FIG. 3(B) for diluted serum IgG 1:1000.

FIGS. 4(A) and (B) show the binding affinity of the anti-Globo H IgG and IgM antibodies with Globo H. FIG. 4(A) shows binding affinity of anti-Globo H IgG antibodies with Globo H. FIG. 4(B) shows binding affinity of anti-Globo H IgM antibodies with Globo H.

FIGS. 5(A) and (B) show that mice immunized with 2 μg or 8 μg of MZ-11-Globo H glycopeptide prepared as in Example 1, plus QS21 adjuvant (2 μg) exhibit high-titer of anti-Globo H IgG and IgM with immune boost effect. MZ-11-4KA-Globo H is a quadruple Globo H conjugated vaccine, which has four Globo H antigens conjugated via four triazole moieties to four consecutive lysine residues at the C-terminal end of a single pan-DR epitope (“PADRE”) sequence.

FIG. 6 shows that antibodies in serum from mice vaccinated with MZ-11-Globo H glycopeptide of Example 1, plus adjuvant QS21, bind to Globo H-expressing MCF-7 cells.

FIGS. 7(A) and (B) show that MZ-11-Globo H glycopeptide compound of Example 1 (“M: Globo H-PADRE” in figure key) induces a higher titer of anti-Globo H IgG antibody than is induced by a general carrier protein-Globo H conjugate (“G: Protein carrier Globo H” in figure key). C: control; Q: adjuvant QS21. FIG. 7(A) refers to the results of mouse-anti-Globo H IgG ELISA and FIG. 7(B) refers to the results of mouse-anti-Globo H IgM ELISA.

FIGS. 8(A) and 8(B) shows that antibody titers in individual mouse receiving MZ-11-Globo H glycopeptide compound of Example 1 as vaccine are constantly high, whereas antibody titers in mouse receiving carrier protein-Globo H conjugate (“G”) are variable and most are low. FIG. 8(A) shows the results of the carrier protein-Globo H conjugate (“G”) vaccine Mouse anti-GloboH IgG ELISA. FIG. 8(B) shows the results of MZ-11-Globo vaccine Mouse anti-Globo H IgG ELISA. G1-G10 represent mice No.1-No.10 receiving carrier protein-Globo H conjugate (“G”) vaccine. M1-M10 represent mice No.1-No.10 receiving MZ-11-Globo H glycopeptide vaccine.

FIGS. 9(A) and (B) show that MZ-11-Globo H glycopeptide compound (“M” on plot) induces long-term anti-Globo H IgG induction in mice, whereas general carrier protein-Globo H conjugate (“G” on plot) does not. FIG. 9(A) shows the results of mouse serum anti-Globo H IgG and FIG. 9(B) shows the results of mouse serum anti-Globo H IgM.

FIGS. 10(A) and (B) show that dissected mice (Day 109) receiving the MZ-11-Globo H glycopeptide compound shows constantly long-lived high-titer anti-Globo H IgG antibody, whereas mice receiving general carrier protein-Globo H conjugate (“G” on plot) do not. FIG. 10(A) for D109 mouse serum anti-Globo H IgG and FIG. 10(B) for D109 mouse serum anti-Globo H IgM. G1-G10 represent mice No.1-No.10 receiving carrier protein-Globo H conjugate vaccine. M1-M10 represent mice No.1-N.10 receiving MZ-11-Globo H glycopeptide vaccine.

FIGS. 11(A) and (B) show that a GM2-PADRE glycopeptide induces high-titer anti-carbohydrate IgG antibody. FIG. 11(A) shows results for the induction of IgG and FIG. 11(B) for the induction of IgM.

FIG. 12 shows that mouse treated with MZ-11-Globo H glycopeptide vaccine demonstrated slower tumor growth.

FIG. 13 shows that mouse treated by adoptive transfer of serum from mice immunized with MZ-11-Globo H glycopeptide vaccine showed smaller tumor burden than controls.

FIGS. 14(A) to (H) shows results using polyvalent vaccine compositions comprising mixtures of MZ-11-Globo H, GM2-PADRE, Lewis Y-PADRE conjugates, or SSEA4-PADRE, GM2-PADRE, Lewis Y-PADRE conjugates can induce high-titer of IgG against each of the respective carbohydrate antigen in the mixture. (FIG. 14(A) for Globo H IgG; FIG. 14(B) for Globo H IgM; FIG. 14(C) for GM 2IgG; FIG. 14(D) for GM2 IgM; FIG. 14(E) for LewisY IgG; FIG. 14(F) for LewisY IgM; FIG. 14(G) for SSEA4 IgG and FIG. 14(H) for SSEA4 IgM).

### DETAILED DESCRIPTION

The present disclosure is based, at least in part, on the finding that a glycopeptide conjugate compound of a tumor-associated carbohydrate antigen and the pan-DR epitope (“PADRE”) sequence is capable of eliciting an immune response in a mammal. This immunogenic glycopeptide compound facilitates the activation of both B cells and T cells, thereby resulting in the production of IgM and IgG antibodies that specifically bind to the carbohydrate antigen. Further, the immunogenic glycopeptide conjugate compound can be used as a vaccine capable of inducing high-titer anti-carbohydrate IgG antibody for treating cancer, particularly cancers that express tumor-associated carbohydrate antigens. More particularly, polyvalent vaccines based on the glycopeptide conjugate compounds disclosed herein can elicits high-titer polyvalent anti-carbohydrate IgG antibodies for treating cancer, particularly cancers that express tumor-associated carbohydrate antigens.

Therefore, in one aspect, the present disclosure is directed to the immunogenic glycopeptide compounds disclosed herein (e.g., the compounds of structural formulae (I), (II), and (III)). Moreover, the immunogenic glycopeptide compounds according to the present disclosure can be used in methods the prevention and/or treatment of cancer. Further, the compounds can be manufactured as a medicament, e.g., as part of a pharmaceutical composition. Thus, the present immunogenic glycopeptide compounds and pharmaceutical compositions comprising the same can also be used in a method for treating and/or preventing cancer. Accordingly, the present disclosure also contemplates a method for treating cancer in a subject suffering therefrom comprising administering to said subject a therapeutically effective amount of the immunogenic glycopeptide compound or pharmaceutical composition as defined herein. In addition, such methods contemplate the use of the pharmaceutical compositions comprising the immunogenic glycopeptide(s) as vaccines for the prevention and/or treatment of cancer.

Definitions

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “antigen” as used herein is defined as a substance capable of eliciting an immune response. Said immune response may involve either antibody production, or the activation of specific immunologically-competent cells, or both. As used herein, the term “immunogen” refers to an antigen capable of inducing the production of an antibody. Also, the term “immunogenicity” generally refers to the ability of an immunogen or antigen to stimulate an immune response.

The term “epitope” refers to a unit of structure conventionally bound by an immunoglobulin V_{H}/V_{L} pair. An epitope defines the minimum binding site for an antibody, and thus represent the target of specificity of an antibody.

As used herein, the term “glycopeptide” refers to a compound in which carbohydrate is covalently attached to a peptide or oligopeptide.

Unless specified otherwise, in the peptide notation used herein, the left-hand direction is the amino-terminal (N-terminal) direction and the right-hand direction is the carboxy-terminal (C-terminal) direction, in accordance with standard usage and convention.

“Percentage (%) amino acid sequence identity” with respect to the amino acid sequences identified herein is defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). Specifically, the percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in A or B, whichever is shorter.

As discussed herein, minor variations in the amino acid sequences of proteins/polypeptides are contemplated as being encompassed by the presently disclosed and claimed inventive concept(s), providing that the variations in the amino acid sequence maintain at least 90%, such as at least 91%, 92%, 93%, 94%, 95%, 96%, 97%, 98% and 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic lysine, arginine, histidine; (3) nonpolar alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of proteins/polypeptides can be readily prepared by those of ordinary skill in the art. Preferred amino- and carboxy-termini of fragments or analogs occur near boundaries of functional domains.

Unless contrary to the context, the term “treatment” are used herein broadly to include a preventative (e.g., prophylactic), curative, or palliative measure that results in a desired pharmaceutical and/or physiological effect. Preferably, the effect is therapeutic in terms of partially or completely curing or preventing cancer. Also, the terms “treatment” and “treating” as used herein refer to application or administration of the present immunogenic glycopeptide, antibody, or pharmaceutical composition comprising any of the above to a subject, who has cancer, a symptom of cancer, a disease or disorder secondary to cancer, or a predisposition toward cancer, with the purpose to partially or completely alleviate, ameliorate, relieve, delay onset of, inhibit progression of, reduce severity of, and/or reduce incidence of one or more symptoms or features of cancer. Generally, a “treatment” includes not just the improvement of symptoms or decrease of markers of the disease, but also a cessation or slowing of progress or worsening of a symptom that would be expected in absence of treatment. The term “treating” can also be used herein in a narrower sense which refers only to curative or palliative measures intended to ameliorate and/or cure an already present disease state or condition in a patient or subject.

The term “preventing” as used herein refers to a preventative or prophylactic measure that stops a disease state or condition from occurring in a patient or subject. Prevention can also include reducing the likelihood of a disease state or condition from occurring in a patient or subject and impeding or arresting the onset of said disease state or condition.

As used herein, the term “therapeutically effective amount” refers to the quantity of an active component which is sufficient to yield a desired therapeutic response. A therapeutically effective amount is also one in which any toxic or detrimental effects of the compound or composition are outweighed by the therapeutically beneficial effects.

As used herein, a “pharmaceutically acceptable carrier” is one that is suitable for use with the subjects without undue adverse side effects (such as toxicity, irritation, and allergic response) commensurate with a reasonable benefit/risk ratio. Also, each carrier must be “acceptable” in the sense of being compatible with the other ingredients of the pharmaceutical composition. The carrier can be in the form of a solid, semi-solid, or liquid diluent, cream or a capsule. The carrier must be “acceptable” in the sense of being compatible with the other ingredients of the formulation, and is selected to minimize any degradation of the active agent and to minimize any adverse side effects in the subject.

As used herein, the term “adjuvant” refers to an immunological agent that modifies the effect of an immunogen, while having few if any direct effects when administered by itself. It is often included in vaccines to enhance the recipient's immune response to a supplied antigen, while keeping the injected foreign material to a minimum. Adjuvants are added to vaccines to stimulate the immune system's response to the target antigen, but do not in themselves confer immunity

As used herein, the term “subject” refers to a mammal including the human species that is treatable with antibody. The term “subject” is intended to refer to both the male and female gender unless one gender is specifically indicated.

Immunogenic Glycopeptide Compounds

The present disclosure provides immunogenic glycopeptide compounds, wherein the compounds have the structural formula (I)

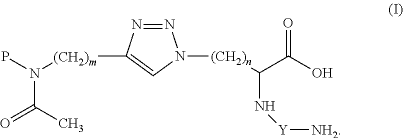

In the structural formula (I), P is a carbohydrate antigen selected from Globo H, SSEA4, GD2, GD3, GM2, fucosyl GM1, LewisY Le(y), sialyl-Le(x), sialyl-Le(a), TF, Tn and sialyl-Tn. The carbohydrate antigen is connected to the triazole moiety via an N-acetyl group and alkyl linker of from 1 to 4 carbons (m=1 to 4). Y is a pan-DR epitope (also referred to herein as “PADRE”) sequence. The pan-DR epitope is connected to the triazole moiety via an alkyl linker of from 1 to 5 carbons (n=1 to 5), wherein the alkyl linker is attached at the alpha carbon of an amino acid residue.

The immunogenic glycopeptide compounds of structural formula (I) feature a triazole moiety that covalently links the carbohydrate antigen to the pan-DR epitope. As such, glycopeptide compounds of formula (I) can be formed using the Cu(I)-mediated Huisgen “click reaction” as shown in Scheme 1 and further exemplified in Example 1 below.

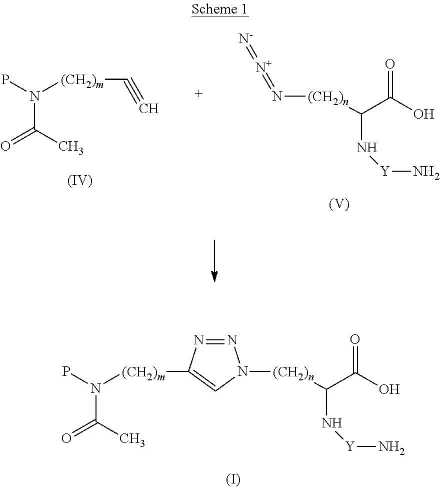

As shown in Scheme 1, the pan-DR epitope is modified with an azide group, as depicted in the compound of formula (V). Typically, this can be an azido-modified amino acid residue introduced at the C-terminus of the pan-DR epitope sequence using standard automated peptide synthesis. Exemplary azido-modified amino acid residues that can be used to prepare azido-modified pan-DR epitopes include but are not limited to azido-lysine, azido-butyl-alanine, and azido-phenylalanine. Both azido-lysine and azido-butyl-alanine have side chains that introduce a four carbon alkyl linker when used to prepare a compound of structural formula (I).

As depicted in Scheme 1, the carbohydrate antigen (“P”) is modified with an N-acetyl propargyl group, as depicted in the compound of formula (IV). Synthetic methods for introducing N-acetyl propargyl groups to carbohydrates are known in the art and many propargyl modified carbohydrate antigens (e.g., Globo H-b-N-acetyl propargyl) are commercially available.

The propargyl group of the carbohydrate antigen reacts efficiently with the azide group of the pan-DR epitope to yield the triazole moiety and the glycopeptide compound of structural formula (I). It was previously thought that the rigid triazole moiety would have its own immunogenicity that would further suppress the low immunogenicity of a linked carbohydrate antigen (see e.g., Buskas et al., “Immunotherapy for Cancer: Synthetic Carbohydrate-based Vaccines,” Chem. Commun. (Camb). 2009 Sep. 28; (36): 5335-5349). Thus, a surprising result of the present disclosure, as demonstrated in the examples herein, is that the glycopeptide compounds of structural formula (I) exhibit specific and high immunogenicity for the carbohydrate antigen, and furthermore elicit high titers of IgG antibodies.

“Globo H” is a hexasaccharide, which is a member of a family of antigenic carbohydrates that are highly expressed on a various types of cancers, especially cancers of breast, prostate and lung (Dube D H, Bertozzi C R, (2005) Glycans in cancer and inflammation. Potential for therapeutics and diagnostics. Nat Rev Drug Discov 4:477-488). It is expressed on the cancer cell surface as a glycolipid and possibly as a glycoprotein (Livingston P O, (1995) Augmenting the immunogenicity of carbohydrate tumor antigens. Cancer Biol 6:357-366). The structure of Globo H is as follows.

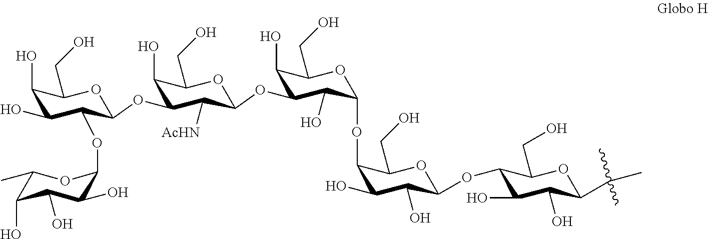

“GD2” is a disialoganglioside expressed on tumors of neuroectodermal origin, including human neuroblastoma and melanoma, with highly restricted expression on normal tissues, principally to the cerebellum and peripheral nerves in humans (Wierzbicki, Andrzej et al., (2008). “Immunization with a Mimotope of GD2 Ganglioside Induces CD8+ T Cells That Recognize Cell Adhesion Molecules on Tumor Cells”. Journal of Immunology 181 (9): 6644-6653). The structure of GD2 is as follows.

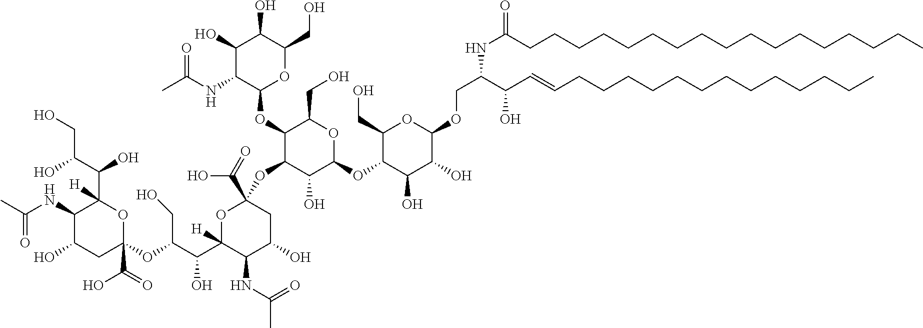 [SMILES: CCCCCCCCCCCCC/C=C/[C@H](O)[C@H](CO[C@@H]1O[C@H](CO)[C@@H](O[C@@H]2O[C@H](CO)[C@H](O[C@@H]3O[C@H](CO)[C@H](O)[C@H](O)[C@H]3NC(C)=O)[C@H](O[C@]3(C(=O)O)C[C@H](O)[C@@H](NC(C)=O)[C@H]([C@H](O)[C@@H](CO)O[C@]4(C(=O)O)C[C@H](O)[C@@H](NC(C)=O)[C@H]([C@H](O)[C@H](O)CO)O4)O3)[C@H]2O)[C@H](O)[C@H]1O)NC(=O)CCCCCCCCCCCCCCCCC]

The “GM2” is a type of ganglioside. G refers to ganglioside, the M is for monosialic (as in it has one sialic acid), and 2 refers to the fact that it was the second monosialic ganglioside discovered (Guetta E, Peleg L (2008). “Rapid Detection of Fetal Mendalian Disorders: Tay-Sachs Disease”. Methods Mol. Biol. 444: 147-59). The structure of GM2 is as follows.

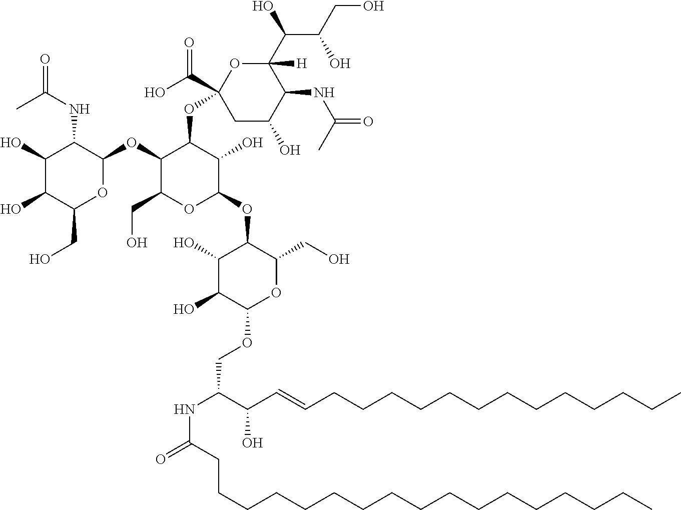 [SMILES: CCCCCCCCCCCCC/C=C/[C@H](O)[C@@H](CO[C@H]1O[C@@H](CO)[C@H](O[C@H]2O[C@@H](CO)[C@@H](O[C@H]3O[C@@H](CO)[C@@H](O)[C@@H](O)[C@@H]3NC(C)=O)[C@@H](O[C@@]3(C(=O)O)C[C@@H](O)[C@H](NC(C)=O)[C@@H]([C@@H](O)[C@@H](O)CO)O3)[C@@H]2O)[C@@H](O)[C@@H]1O)NC(=O)CCCCCCCCCCCCCCCCC]

“SSEA-4”, a sialyl-glycolipid, has been commonly used as a pluripotent human embryonic stem cell marker, and its expression is correlated with the metastasis of some malignant tumors.

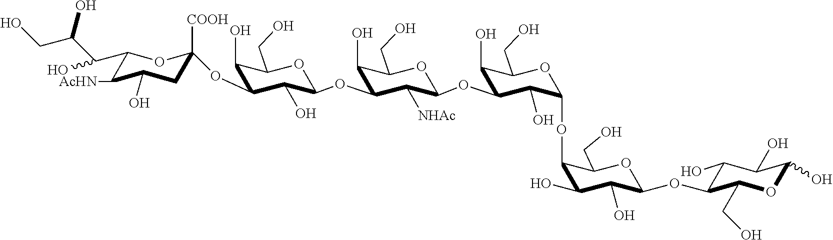

The Lewis antigen system is a human blood group system based upon genes on chromosome 19 p13.3 (FUT3 or Lewis gene) and 19q13.3, (FUT2 or secretor gene). There are two main types of Lewis antigens, Lewis a (Le-a) and Lewis b (Le-b) (Mais D D. ASCP Quick Compendium of Clinical Pathology, 2nd Ed. Bethesda: ASCP Press, 2008). The Lewis(y) antigen is an oligosaccharide containing two fucoses, and is expressed variously in 75% of ovarian tumors, where its high expression level predicts poor prognosis (Liu J J et al., Oncid Rep 2010 March; 23(3):833-41). The structure of LewisY is as follows.

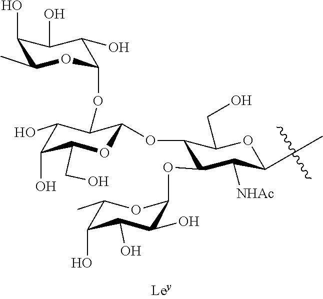

The sialyl-Tn antigen (STn) is a short O-glycan containing a sialic acid residue n2,6-linked to GalNAcα-O-Ser/Thr. The structure of STn is as follows.

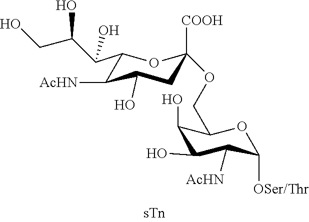

The pan-DR epitope sequence is a non-natural sequence engineered to introduce anchor residues for different known DR-binding motifs. For example, X (cyclohexylalanine) in position 3 is an aliphatic residue corresponding to the position 1 of DR-binding motif, T in position 8 is a non-charged hydroxylated residue corresponding to position 6 of DR-binding; while A in position 11 is a small hydrophobic residue corresponding to position 9 of the DR-binding motif. Generally, substituting one residue with another residue of substantially the same chemical and/or structural property, e.g., substituting X (cyclohexylalanine) with aromatic F (phenylalanine) or Y (tyrosine), will not significantly affect the binding affinity of the sequence.

A range of pan-DR epitope sequences are known in the art and the present disclosure contemplates that these may be used in an immunogenic glycopeptide compound of structural formula (I). (See e.g., pan-DR epitope sequences disclosed in US patent publication US2005/0049197A1, which is hereby incorporated by reference herein.)

In one embodiment of the immunogenic glycopeptide compounds of structural formula (I), the pan-DR epitope comprises an amino acid sequence at least 80% identical to AKXVAAWTLKAAA (SEQ ID NO: 1), wherein X is an amino acid residue selected from cyclohexylalanine, phenylalanine, and tyrosine. In some embodiments, the pan-DR epitope amino acid sequence is at least 81%, 82%, 83%, 84%, 85%, 86%, 87%, 88%, 89%, 90%, 91%, 91%, 92%, 93%, 94%, 95%, 96%, 97%, 98%, or 99% identical to SEQ ID NO: 1. According to one embodiment, the pan-DR epitope amino acid sequence is identical SEQ ID NO: 1.

In some embodiments, the pan-DR epitope and has at least 10 consecutive amino acid residues that are identical to the 13 amino acid sequence of AKXVAAWTLKAAA (SEQ ID NO: 1).

In one embodiment of the pan-DR epitope sequence, the C-terminal alanine (A) residue of SEQ ID NO: 1 can be omitted. In certain embodiments, the N-terminal alanine (A) residue of SEQ ID NO: 1, or the first two N-terminal residues, alanine (A) and lysine (K) of SEQ ID NO: 1 can be omitted. In one embodiment, the pan-DR epitope sequence is the amino acid sequence of SEQ ID NO: 1 with the two N-terminal residues (A and K) and the C-terminal A residue deleted.

In some embodiments of the immunogenic glycopeptide compound of structural formula (I), the pan-DR epitope consists of the amino acid sequence AKXVAAWTLKAAA (SEQ ID NO: 1) or the amino acid sequence AKXVAAWTLKAA (SEQ ID NO: 2).

In some embodiments of the immunogenic glycopeptide compound of structural formula (I), the amino acid residue X is cyclohexylalanine.

Although it is contemplated that the length of the alkyl linkers of the compound of structural formula (I) can be varied without a loss of immunogenicity, in some aspects the compound of structural formula (I) can have m=1, and/or n=4.

It is contemplated in the present disclosure that the immunogenic glycopeptide compounds of structural formula (I) can be formulated with a variety of carbohydrate antigens. In some aspects, the present disclosure provides immunogenic glycopeptide compounds of structural formula (I), wherein the carbohydrate antigen is selected from group consisting of Globo H, SSEA4, GD2, GD3, GM2, fucosyl GM1, LewisY Le(y), sialyl-Le(x), sialyl-Le(a), TF, Tn and sialyl-Tn.

In some embodiments, the disclosure provides immunogenic glycopeptide compounds of structural formula (I), wherein the carbohydrate antigen is Globo H.

In one embodiment, the immunogenic glycopeptide compound has structural formula (II)

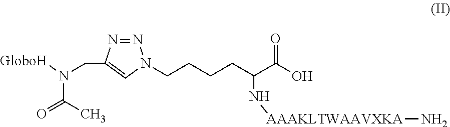

wherein,“GloboH” is the carbohydrate antigen, Globo H, and the pan-DR epitope consists of the amino acid sequence AKXVAAWTLKAAA (SEQ ID NO: 1), wherein X is cyclohexylalanine.

In another embodiment, the immunogenic glycopeptide compound has structural formula (III)

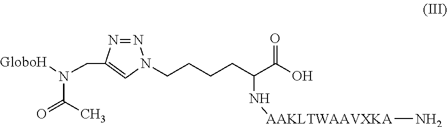

wherein,“GloboH” is the carbohydrate antigen, Globo H, and the pan-DR epitope consists of the amino acid sequence AKXVAAWTLKAA (SEQ ID NO: 2), and wherein X is cyclohexylalanine.

Both the immunogenic glycopeptide compounds of structural formula (II) and (III) can be prepared using the general “click reaction” synthesis method of Scheme 1, which is further exemplified in Example 1 below.

Immunogenic Glycopeptide Compound Pharmaceutical Compositions and Uses Thereof

The immunogenic glycopeptide compounds of the present disclosure are designed to elicit an immune response against certain carbohydrate antigens (e.g., Globo H, GD2, GM2, SSEA 4, Lewis, LewisY, and STn) which are known to be expressed on tumor cells associated with certain cancer types (e.g., breast cancer, ovarian cancer, pancreatic cancer, prostate cancer, colorectal cancer or lung cancer). Accordingly, the present disclosure contemplates the use of the immunogenic glycopeptide compounds disclosed herein, alone and in pharmaceutical compositions, including vaccines and polyvalent vaccines, in methods for preventing and/or treating a cancer in a subject. Generally, the methods for preventing and/or treating cancer in a subject comprise administering to the subject in a therapeutically (or immunogenically) effective amount, the immunogenic glycopeptide compounds disclosed herein, alone or as part of a pharmaceutical compositions.

Thus, in another embodiment the present disclosure provides a pharmaceutical composition comprising a therapeutically effective amount of an immunogenic glycopeptide compound as disclosed herein (e.g., compound of any one of structural formulae (I), (II), or (III) as described above), and a pharmaceutically acceptable carrier and/or an adjuvant, such as an immunogenic adjuvant.

In some aspects of the pharmaceutical composition embodiments disclosed herein, the composition is a vaccine. In some embodiments, the vaccine is a polyvalent vaccine comprising two or more immunogenic glycopeptide compounds as disclosed herein (e.g., compound of structural formula (I)), and each of the of the two or more compounds has a different carbohydrate antigen selected from Globo H, SSEA4, GD2, GD3, GM2, fucosyl GM1, LewisY Le(y), sialyl-Le(x), sialyl-Le(a), TF, Tn and sialyl-Tn. In some embodiments of the polyvalent vaccine composition, the two or more compounds comprise the carbohydrate antigens: Globo H, SSEA4, GD2, GD3, GM2, fucosyl GM1, LewisY Le(y), sialyl-Le(x), sialyl-Le(a), TF, Tn and sialyl-Tn.

As described above, in addition to the immunogenic glycopeptide compounds, the pharmaceutical compositions (including vaccines) comprises a pharmaceutically acceptable carrier and/or an adjuvant. The pharmaceutical composition may further comprise one or more pharmaceutically acceptable additives, including binders, flavorings, buffering agents, thickening agents, coloring agents, anti-oxidants, diluents, stabilizers, buffers, emulsifiers, dispersing agents, suspending agents, antiseptics and the like.

The choice of a pharmaceutically-acceptable carrier to be used in conjunction with a pharmaceutical composition comprising one of the immunogenic glycopeptide compounds of the present disclosure is basically determined by the way the composition is to be administered. The pharmaceutical composition of the present invention may be administered orally or subcutaneous, intravenous, intrathecal or intramuscular injection.

Injectables for administration can be prepared in sterile aqueous or non-aqueous solutions, suspensions, and emulsions. Examples of non-aqueous solvents include, but are not limited to, propylene glycol, polyethylene glycol, vegetable oils such as olive oil, and injectable organic esters such as ethyl oleate. Illustrative examples of aqueous carriers include water, alcoholic/aqueous solutions, emulsions or suspensions, including saline and buffered media. Common parenteral vehicles include sodium chloride solution, Ringer's dextrose, dextrose and sodium chloride, lactated Ringer's, or fixed oils; whereas intravenous vehicles often include fluid and nutrient replenishers, electrolyte replenishers (such as those based on Ringer's dextrose), and the like.

In some embodiments, the pharmaceutical composition may comprise an adjuvant, such as an immunogenic adjuvant. An immunogenic adjuvant is a compound that, when combined with an antigen, increases the immune response to the antigen as compared to the response induced by the antigen alone. For example, an immunogenic adjuvant may augment humoral immune responses, cell-mediated immune responses, or both. Exemplary immunogenic adjuvants useful as adjuvants in the pharmaceutical compositions of the present disclosure include, but are not limited to: mineral salts, polynucleotides, polyarginines, ISCOMs, saponins, monophosphoryl lipid A, imiquimod, CCR-5 inhibitors, toxins, polyphosphazenes, cytokines, immunoregulatory proteins, immunostimulatory fusion proteins, co-stimulatory molecules, and combinations thereof. Mineral salts include, but are not limited to, AIK(SO₄)₂, AlNa(SO₄)₂, AlNH(SO₄)₂, silica, alum, Al(OH)₃, Ca₃(PO4)₂, kaolin, or carbon. Useful immunostimulatory polynucleotides include, but are not limited to, CpG oligonucleotides with or without immune stimulating complexes (ISCOMs), CpG oligonucleotides with or without polyarginine, poly IC or poly AU acids. Toxins include cholera toxin. Saponins include, but are not limited to, QS21, QS17 or QS7. Also, examples of are muramyl dipeptides, N-acetyl-muramyl-L-threonyl-D-isoglutamine (thr-DMP), N-acetyl-nornuramyl-L-alanyl-D-isoglutamine, N-acetylmuramyul-L-alanyl-D-isoglutaminyl-L-alanine-2-(1′2′-dipalmitoyl-sn-glycero-3-hydroxphosphoryloxy)-ethylamine, RIBI (MPL+TDM+CWS) in a 2 percent squalene/TWEEN 80 emulsion, lipopolysaccharides and its various derivatives, including lipid A, Freund's Complete Adjuvant (FCA), Freund's Incomplete Adjuvants, Merck Adjuvant 65, polynucleotides (e.g., poly IC and poly AU acids), wax D from Mycobacterium tuberculosis, substances found in Corynebacterium parvum, Bordetella pertussis, and members of the genus Brucella, Titermax, Quil A, ALUN, Lipid A derivatives, choleratoxin derivatives, HSP derivatives, LPS derivatives, synthetic peptide matrixes or GMDP, Montanide ISA-51 and QS-21, CpG oligonucleotide, poly I:C, and GMCSF.

Combinations of adjuvants can also be used. In some embodiments of the pharmaceutical compositions disclosed herein, the adjuvant is aluminum salts (such as aluminum phosphate and aluminum hydroxide), calcium phosphate, polyinosinic-polycytidylic acid (poly I:C), CpG motif, and saponins (such as Quil A or QS21). In one embodiment, the adjuvant is aluminum hydroxide and/or QS21.

In another aspect, the present invention provides a method for preventing and/or treating a cancer, comprises administering an effective amount of an immunogenic glycopeptide compound described herein (e.g., compound of any one of structural formulae (I), (II), or (III)) or a derivative thereof to a subject. As illustrated in the various working examples presented below, immunizing adult C57BL/6 mice (weight 20-25 grams) with about 2 μg to 54 μg of the immunogenic glycopeptide of structural formula (II) elicits a desired immune response. Hence, in certain embodiments of the present disclosure, the therapeutically effective amount of the immunogenic glycopeptide for mice could be expressed as 0.08-27 mg/kg body weight. The therapeutically effective amount for a human subject can be estimated from the animal doses according to various well-established standards or conversion means. For example, the “Guidance for Industry Estimating the Maximum Safe Starting Dose in Initial Clinical Trials for Therapeutics in Adult Healthy Volunteers” by Food and Drug Administration of U.S. Department of Health and Human Services provides several conversion factors for converting animal doses to human equivalent doses (HEDs). For mice weighted between 11 to 34 grams, to convert the therapeutically effective mouse dose (in mg/kg) to HED (in mg/kg) for a 60 kg adult human, the mouse dose is multiplied by 0.081.

In the instant case, the therapeutically effective amount of the immunogenic glycopeptide compound of structural formula (II) for an adult human subject is 0.06-2.2 mg/kg body weight. Thus, in some embodiments, the therapeutically effective amount of an immunogenic glycopeptide compound to use in the methods of the present disclosure for preventing and/or treating cancer in a human subject is at least 1 mg/kg.

According to various embodiments of the present disclosure, the cancers that can be treated and/or prevent by using the immunogenic glycopeptide compounds, or the pharmaceutical composition comprising the same, in the methods of treatment described herein are tumor-associated carbohydrate-expressing cancers. Preferably, the tumor-associated carbohydrate-expressing cancer is breast cancer, ovarian cancer, pancreatic cancer, prostate cancer, colorectal cancer or lung cancer.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Example 1

### Preparation of Immunogenic Glycopeptide Compound, MZ-11-Globo H

This example illustrates the synthesis of MZ-11-Globo H, an immunogenic glycopeptide compound of structural formula (II), wherein the carbohydrate antigen is Globo H, and the pan-DR epitope consists of the amino acid sequence AKXVAAWTLKAAA (SEQ ID NO: 1), wherein X is cyclohexylalanine.

Briefly, the method of preparation involves a Cu(I)-catalyzed Huisgen click reaction between the pan-DR epitope of sequence AKXVAAWTLKAAA (SEQ ID NO: 1), wherein X is a cyclohexylalanine residue and which also has an additional C-terminal azido-lysine residue, and Globo H-b-acetyl propargyl, as shown in Scheme 2.

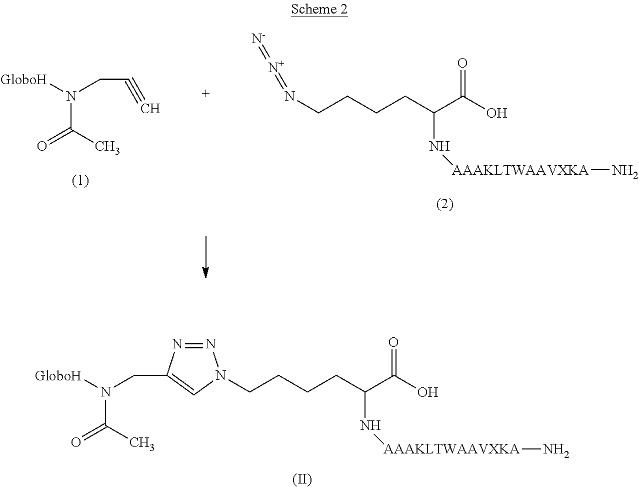

As shown in Scheme 2, the azide group of the C-terminal azido-lysine residue reacts with the propargyl group to yield the triazole moiety that covalently links the pan-DR epitope to the Globo H carbohydrate antigen.

5 mg of Globo H-b-N-acetyl propargyl of compound (1) (Carbosynth Ltd., England) was dissolved in 1 ml of distilled water, and 5.5 mg of the azide-modified pan-DR epitope of compound (2) was dissolved in 110 μl of DMSO. The azide-modified pan-DR epitope of compound (2) is the 13-mer amino acid sequence of SEQ ID NO:1, wherein X is an cyclohexylalanine residue, and with an azido-lysine amino acid residue added to the C-terminus. As shown in Scheme 2, it is the side-chain of the lysine that forms four carbon alkyl chain to the triazole moiety. Compound (2) was prepared using standard solid-phase automated peptide synthesis (Kelowan International Scientific, Inc.; Taiwan). For click reaction, 1 μmole each of compound (1) and compound (2) were first mixed and added with distilled water to a final volume of 500 μl. Then 500 μl of t-butanol (Sigma), 200 μl of 100 mM CuSO4.5H₂O (Sigma), and 160 μl of 500 mM fresh prepared Na-L-ascorbate (Sigma) were sequentially added under magnetic stirring. The mixture was incubated overnight with stirring at room temperature, followed by addition of 50 μl of 27% ammonium hydroxide (Sigma). The product Globo H glycopeptide compound of formula (II), wherein X is cyclohexylalanine, referred to herein as “MZ-11-Globo H,” was further diluted with one volume of distilled water and stored at 4° C.

### Example 2

### Production of Monoclonal Antibodies Against Globo H

Adult female C57BL/6 mice (n=3 each group; 5 weeks old; average weight 16-20 grams; purchased from Biolasco, Taiwan) were immunized by subcutaneous injection with 6 μg of the MZ-11-Globo H glycopeptide of Example 1, and 50 μl of complete Freund's adjuvant (CFA; from Sigma). Four immunizations were given at a 2-week interval. Three days after the fourth immunization, immunized splenocytes were harvest and washed with serum-free medium. Subsequently, 1×10⁸ of single cell suspended splenocytes were mixed with 2×10⁷ of FO cells, and cell fusion was performed in 1 ml of 50% PEG 1500 solution (Roche) at 37° C. followed by drop-wise addition of 13 ml of warmed RPMI medium (Gibco). Fused cells were centrifuged and washed twice with complete medium. Cells were then re-suspended in complete medium with 1×BM-Conditioned H1 Hybridoma cloning supplement (Roche) and seeded into 96-well plates. For target specific B cell-myeloma cells fusion, immunized splenocytes were incubated with Globo H-biotin (10 μg/ml) in serum-free RPMI medium for 3 hours at 4° C. After being washed three times with the same medium, Globo H-binotin-bearing cells were resuspended at a concentration of 1×10⁸ cells/ml and incubated with streptavidin (50 μg/ml) for 30 minutes at 4° C. Meanwhile, FO cells were incubated with 50 μg/ml of NHS-biotin for 1 hour at 4° C. Both treated cells were then washed three times with serum-free RPMI medium. Then, 1×10⁸ splenocytes and 2×10⁷ FO cells were mixed together, and chemical cell fusion was performed as describe above. After cell fusion, cells were cultured in RPMI 1640 medium containing 1×HAT medium (Gibco) for further selection.

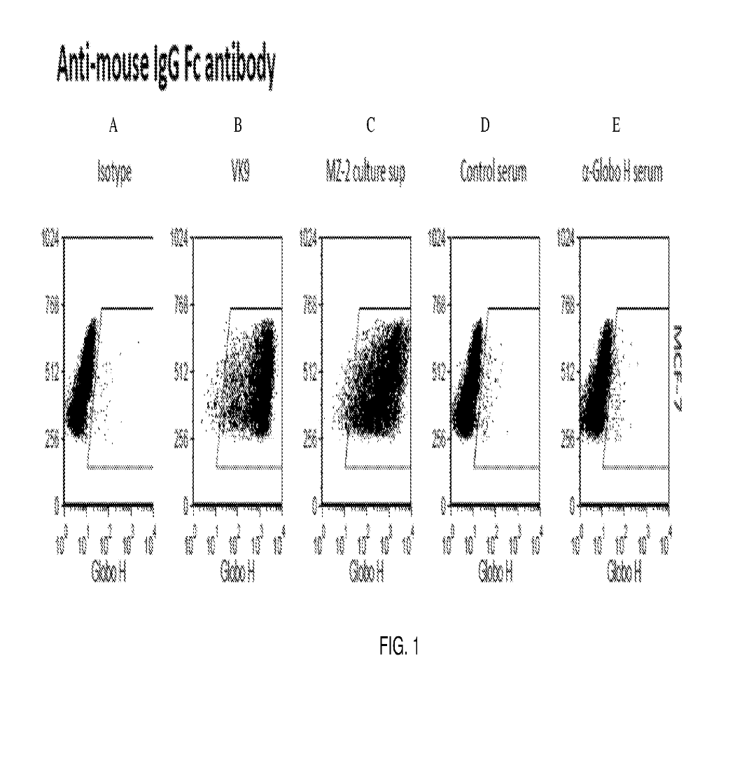

Monoclonal antibody-producing hybridoma cell lines were screened through limited dilution by ELISA assay on plate coated with Globo H-biotin antigen. Five clones (named MZ-1 to MZ-5, respectively) capable of secreting high-titers of anti-Globo H IgG or IgM antibodies were obtained. Supernatants from these hybridoma lines were also subjected to cell binding assay. Briefly, 100 μl of the supernatant from the hybridoma culture was incubated with 2×10⁵ of MCF-7 cells and then analyzed by flow cytometry with appropriate fluorescent secondary antibody mentioned below. The cells were washed once with 2 ml of 1×PBS. After centrifugation, the wash buffer was discarded and cells were resuspended in 100 μl of 1:100 diluted PE anti-mouse IgG-Fc (Jackson immunoresearch) or 100 μl of 1:100 diluted PE anti-mouse IgM (eBioscience) and incubated again at room temperature for 20 minutes. The cells were washed with PBS and resuspended in 200 μl of 1×PBS after centrifugation. The binding of antibodies with cells were detected by flow cytometry. The results provided in FIGS. 1A to E reveal that the monoclonal antibody produced by MZ-2 hybridoma (see FIG. 1C) (hereinafter, the MZ-2 antibody) had good binding affinity to MCF-7 cells. For comparison purposes, a commercially available anti-Globo H IgG3 antibody (see FIG. 1E), VK9 antibody (see FIG. 1B) (eBioscience), was also analyzed.

### Example 3

### Production of Anti-Globo H IgG and IgM Antibodies

Adult female C57BL/6 mice (5 in each group at 5 weeks old, average weight 16-20 gm; Biolasco, Taiwan, R.O.C.) were injected subcutaneously to abdomen region with the Globo H glycopeptide of structural formula (II) as prepared in Example 1, together with the complete Freund's adjuvant (CFA; from Sigma) as the adjuvant. Three immunizations were given at a 2-week interval; each vaccination contained 2 μg, 6 μg or 18 μg Globo H glycopeptide with 50 μl adjuvant. Serum was collected one week after the last immunization, and then subjected to enzyme-linked immunosorbent assay (ELISA) to measure the production of the anti-Globo H antibody. Serum from naive mice injected with PBS and serum from mice immunized with the adjuvant only were used as negative controls. Sera raised against anti-Globo-H antibodies, MBr 1 (Enzo Life Science; 0.5 μg/ml) or MZ-2 (produced as in Example 2; 1 μg/ml) were used as positive controls.

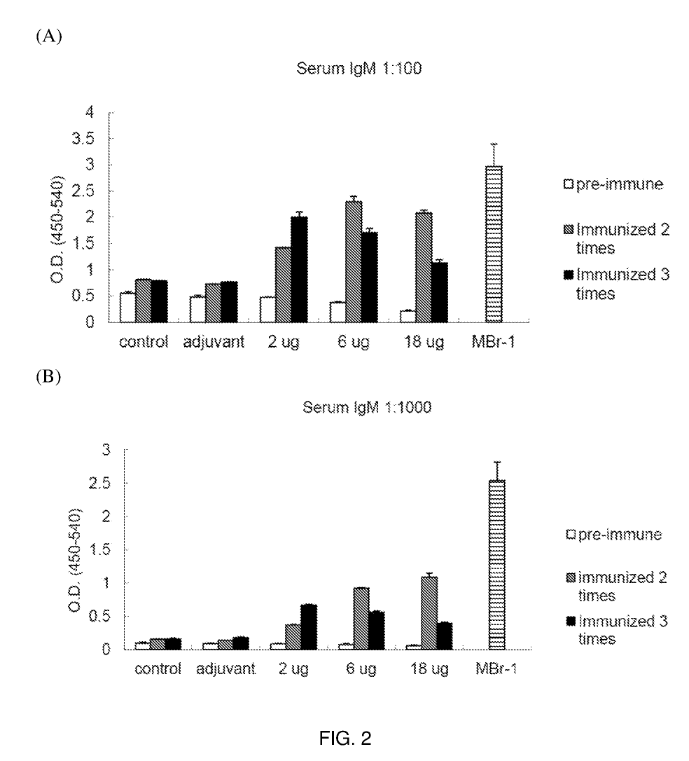

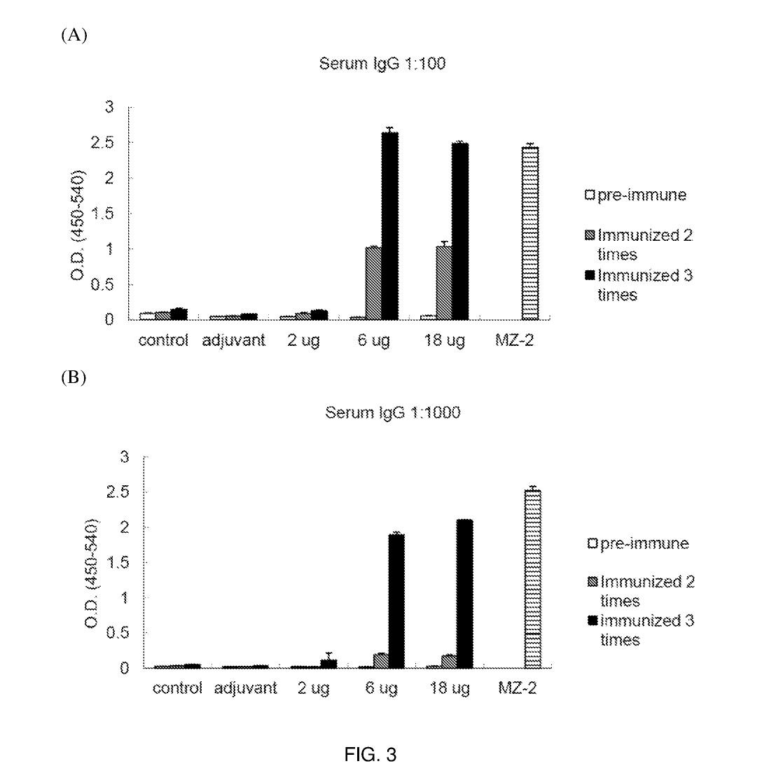

For ELISA, diluted serum (1:100 or 1:1000) from mice immunized with Globo H glycopeptide of formula (II) was added into designated wells of a 96-well ELISA plate and incubated at room temperature for one hour. Wells were then washed six times with 0.1% Tween-20 in 1×PBS. Thereafter, 1:2500 diluted anti-mouse IgG-HRP or anti-mouse IgM-HRP (Jackson Immuno Research) was added to the wells and incubated at room temperature for another one hour, and washed six times with 0.1% Tween-20 in 1×PBS. Color development was performed by incubation of the washed wells with DMT ELISA kit, and stopped by adding 2N H2504. Signals were read and recorded by ELISA reader at O.D. 450 nm (reference: 540 nm). Elisa results are depicted in FIG. 2 (FIG. 2(A) for diluted serum IgM 1:100 and FIG. 2(B) for diluted serum IgM 1:1000) and FIG. 3 (FIG. 3(A) for diluted serum IgG 1:100 and FIG. 3(B) for diluted serum IgG 1:1000).

The data in FIG. 2 indicate that Globo H glycopeptide of formula (II) induced the production of anti-Globo H IgM. For mice immunized with 2 μg Globo H glycopeptide of formula (II), the anti-Globo H IgM titers increased as immunization proceeded.

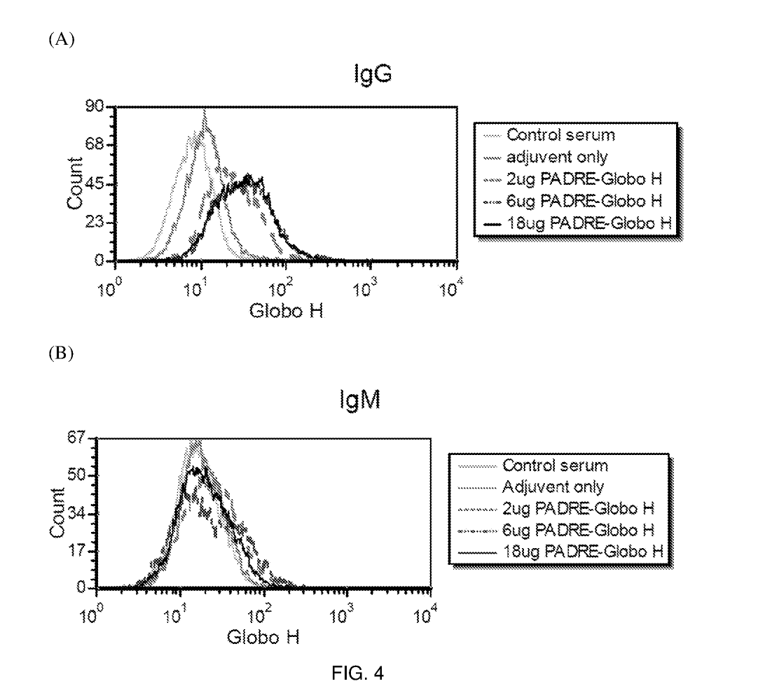

A cell binding assay was performed to elucidate the binding affinity of the anti-Globo H IgG and IgM antibodies with Globo H. Briefly, 100 μl of 1:10 diluted serum or 10 μg/ml of monoclonal antibodies in 1×PBS were incubated with 2×10⁵ of cells at room temperature for 20 minutes. The cells were washed once with 2 ml of 1×PBS. After centrifugation, the wash buffer was discarded and cells were resuspended in 100 μl of 1:100 diluted PE anti-mouse IgG-Fc (Jackson immunoresearch) or 100 μl of 1:100 diluted PE anti-mouse IgM (eBioscience) and incubated again at room temperature for 20 minutes. The cells were washed with PBS and resuspended in 200 μl of 1×PBS after centrifugation. The binding of antibodies with cells were detected by flow cytometry. Results of cell binding assay are summarized in FIG. 4 (FIG. 4(A) for binding affinity of anti-Globo H IgG antibodies with Globo H and FIG. 4(B) for binding affinity of anti-Globo H IgM antibodies with Globo H). As can be seen in FIG. 4, anti-Globo H IgG antibodies obtained from immunizations with the present Globo H glycopeptide of formula (II) displayed excellent recognition of MCF-7 cells which express the Globo H antigen.

### Example 4

### MZ-11 Globo H Glycopeptide Compound Induces High-Titer of Anti-Globo H IgG with Boost Effect

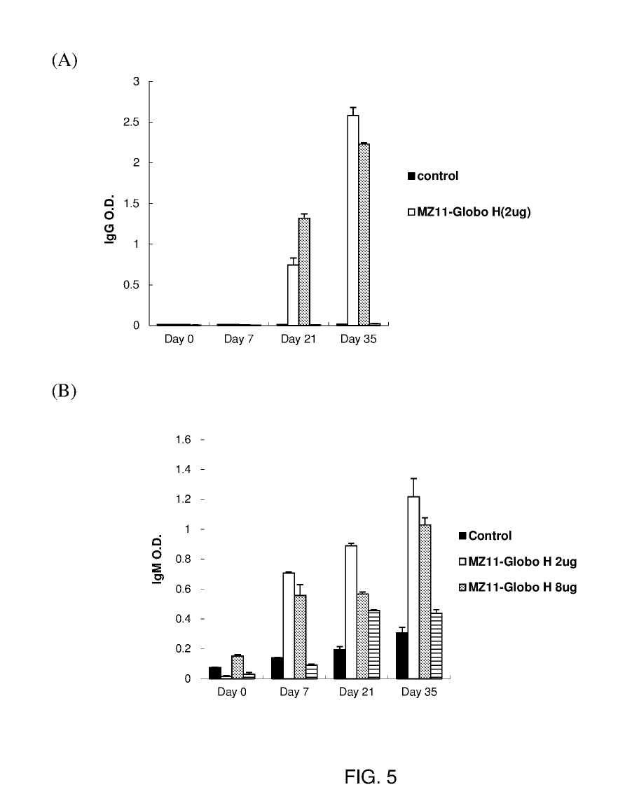

C57BL/6 mice were immunized 3 times with 2 μg or 8 μg of single Globo H conjugated vaccine (i.e., MZ-11-Globo H, made as in Example 1) or 8 μg of a quadruple Globo H conjugated vaccine, which has four Globo H carbohydrate antigens conjugated to four consecutive lysine residues at the C-terminal end of a single pan-DR epitope sequence (referred to as “MZ-11-4KA-Globo H”), plus QS-21 as adjuvant at a 2-week interval. Serum was harvested before and 7 days after each immunization. For ELISA assay, 1 μg of streptavidin (21135, Thermo) was dissolved in 100 μL of 1×PBS and coated on 96-well Costar assay plate (9018, Corning) before loading of biotin-Globo H (0.1 μg/well). The wells were then blocked with 1% BSA in 1×PBS, and incubated with serum 1:1000 diluted in the same blocking solution, followed by washing with 1×PBS-0.1% Tween 20. The bound mouse IgG and IgM were detected using HRP-conjugated goat anti-mouse IgG-Fc (1:5000; 115-035-071, Jackson Immunoresearch) and HRP-conjugated goat-anti-mouse IgM μ chain (1:5000; AP128P, MILLIPORE). The color development was performed by adding 100 uL of NeA-Blue solution (010116-1, Clinical Science Products) and stopped with 50 μL Of 2N sulfuric acid. The O.D. was read at 450 nm subtracted 540 nm as reference. FIG. 5 shows that mice immunized with only 2 μg of the MZ-11-Globo H glycopeptide compound and QS21 adjuvant (2 μg) exhibits high-titer of anti-Globo H IgG and IgM with an immune boost effect.

### Example 5

### IgG in Sera from Mice Immunized with MZ-11-Globo H Efficiently Bind to Globo H-Expression Breast Cancer Cell Line (MCF-7)

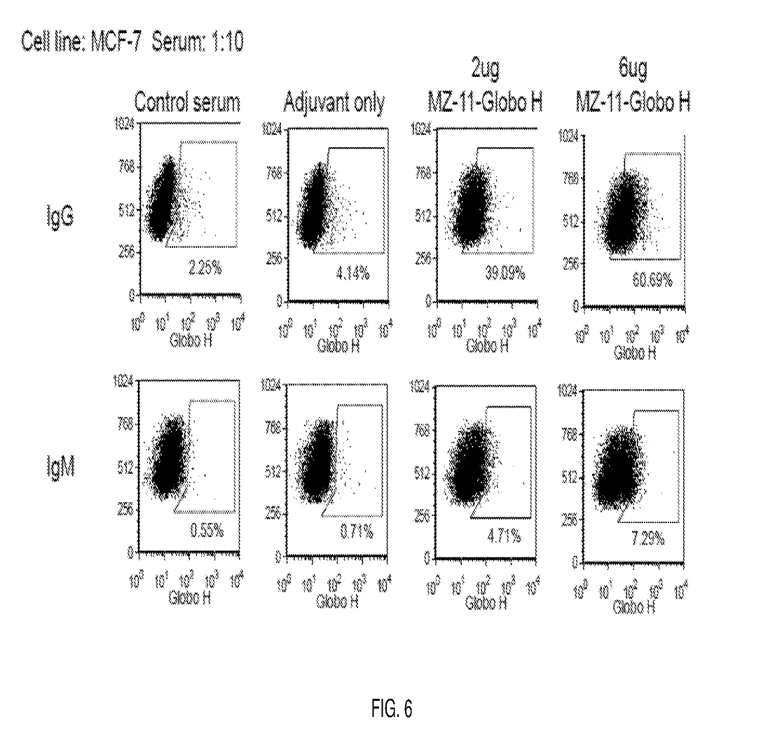

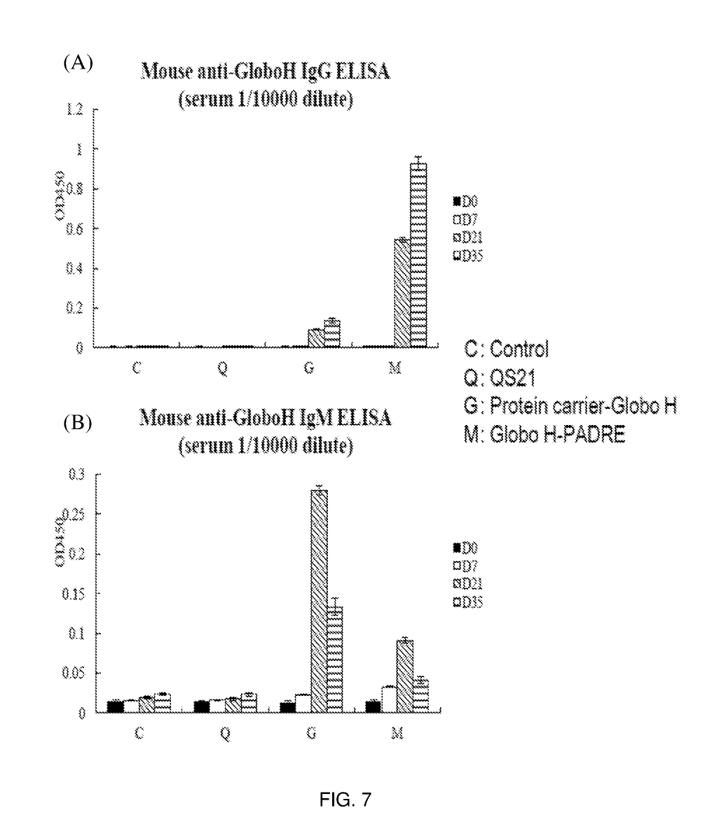

C57BL/6 mice were immunized 3 times with adjuvant alone or 2 μg, 6 μg, or 18 μg of MZ-11-Globo H, made as in Example 1) at a 2-week interval. Anti-serum were harvested 7 days after last immunization. Serum from mice without immunization was collected as control. For FACS, 5×10⁵ of MCF-7 cells were stained with 100 μL of 1:10 diluted serum in flow tube followed by 100 uL of 1:100 diluted PE-conjugated goat anti-mouse IgG-Fc antibody (115-116-071, Jackson immunoresearch) and 1:100 diluted APC-conjugated rat anti-mouse IgM (17-5790-82, eBioscience). The stained cells were analyzed using BD FACSCalibur. FIG. 6 shows that antibodies in serum from mice vaccinated with MZ-11-Globo H (+adjuvant QS21) bind to Globo H-expressing MCF-7 cells.

### Example 6

### MZ-11-Globo H Induces Much Higher Titer Anti-Globo H IgG Than a Carrier Protein-Globo H Conjugate (“G”) with Class Switch

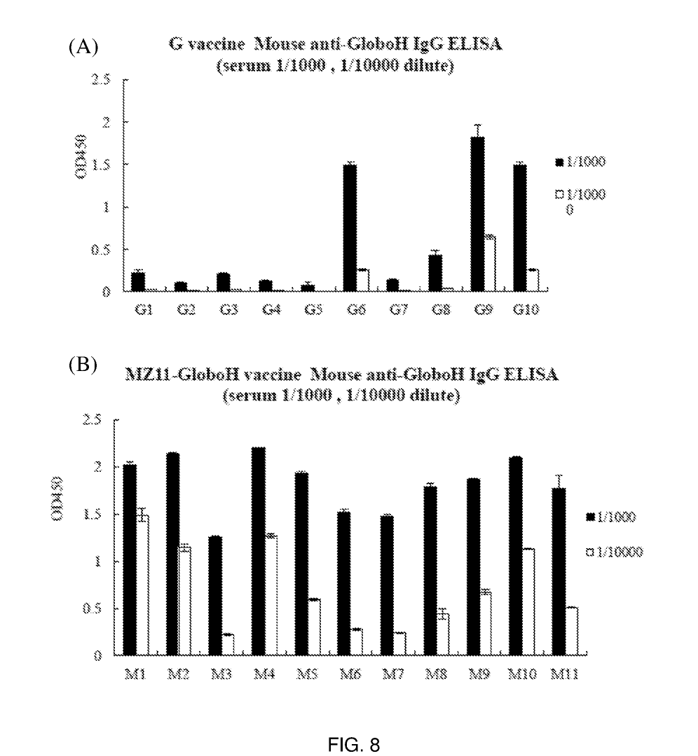

C57BL/6 mice were immunized with adjuvant (QS21 20 μg/mice), 2 μg of general carrier protein-Globo H conjugate vaccine (indicated by “G”), or MZ-11-Globo H glycopeptide prepared as in Example 1 (indicated as “M” or “Globo H-PADRE” in figure key) vaccine at a 2-week interval. Anti-Globo H serum was harvested before and 7 days after each vaccination. The titer of anti-Globo H serum in pooled serum or each mice were detected by ELISA assay with appropriated secondary antibody. FIG. 7 shows that MZ-11-Globo H glycopeptide (“M”) induces higher titer of anti-Globo H IgG antibody than the general carrier protein-Globo H conjugate (“G”) does (or does control “C” or adjuvant QS21 “Q”). FIG. 8 shows that antibody titers in individual mouse receiving g MZ-11-Globo H glycopeptide are constantly high, whereas antibody titers in mouse receiving the carrier protein-Globo H conjugate are variable and most are low and it represents that MZ-11-Globo H glycopeptide stably induces much higher titers of anti-Globo H IgG antibodies in individual mouse.

### Example 7

### MZ-11-Globo H Glycopeptide Induces Long-Lived Anti-Globo H IgG Antibody and B Cell Memory Responses

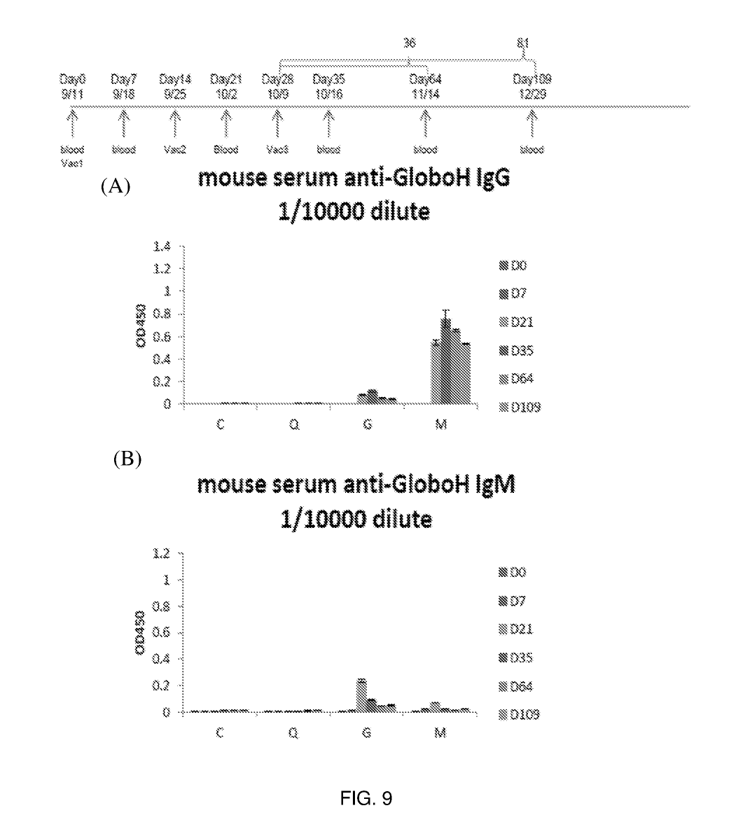

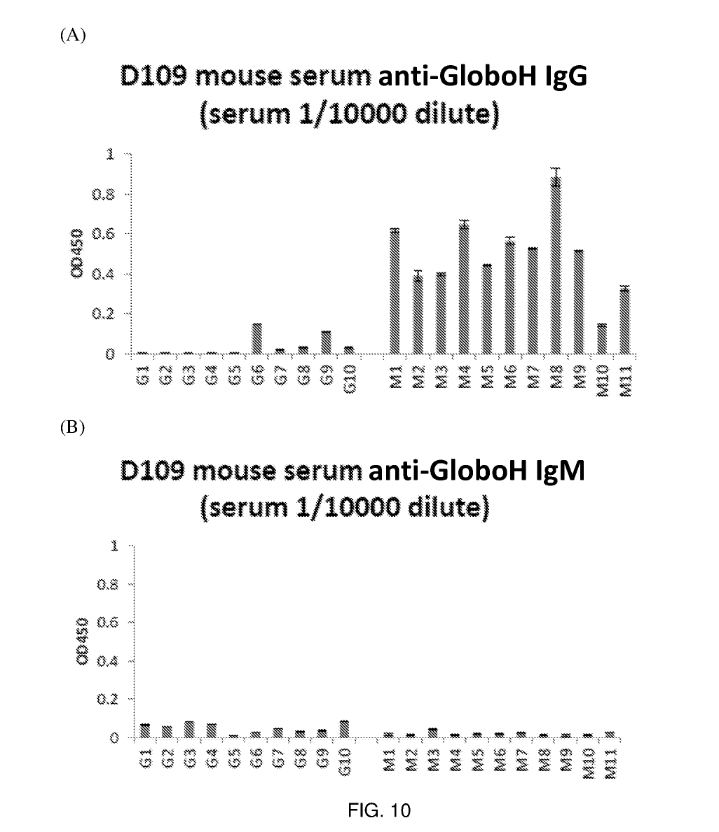

Anti-Globo H serum was harvested on 36 and 81 days after last vaccination (D64 and D109). The titer of anti-Globo H antibodies and the titers of anti-Globo H serum in pooled serum for each mouse were detected by ELISA assay with appropriated secondary antibody with 1/10000 dilution. FIG. 9 shows that MZ-11-Globo H glycopeptide (“M”) induces long-term anti-Globo H IgG, whereas the general carrier protein-Globo H conjugate (“G”) does not. FIG. 10 further shows that dissection of individual mice receiving MZ-11-Globo H glycopeptide showed constantly long-lived high-titer anti-Globo H IgG antibody. Very low level of anti-Globo H IgG antibody was noted in mice receiving the carrier protein-Globo H conjugate.

### Example 8

### Carbohydrate-PADRE Glycopeptide Induces High-Titer Anti-carbohydrate IgG Antibody (GM2 as Example)

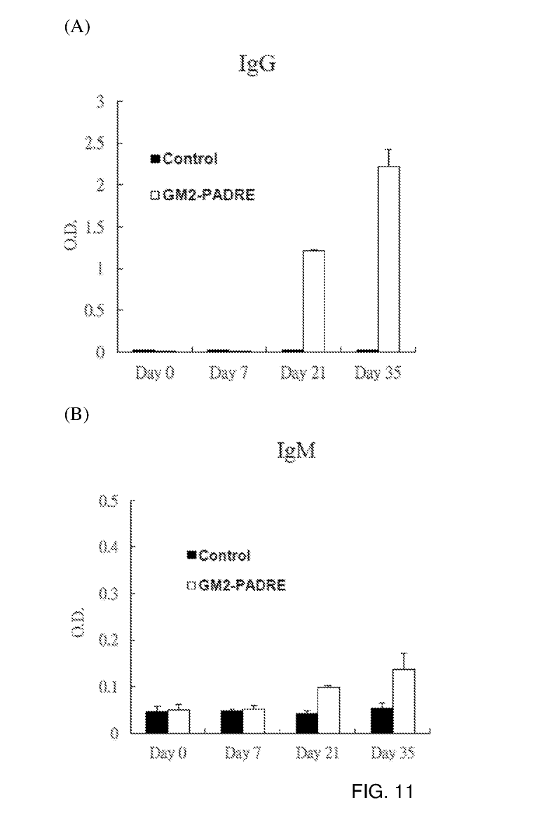

C57BL/6 mice were immunized with adjuvant (QS21 20 μg/mice) or a GM2-PADRE conjugate vaccine with adjuvant (QS-21 20 μg/mice) at a 2-week interval. Anti-GM2 serum was harvested before and 7 days after each vaccination. The titer of anti-GM2 serum in pooled serum or each mice were detected by ELISA assay with appropriated secondary antibody. FIG. 11 shows that the GM2-PADRE glycopeptide conjugate also induces a high-titer anti-carbohydrate IgG antibody.

### Example 9

### Anti-tumor Effect of MZ-11-Globo H Glycopeptide Vaccine in Immuno-Competent Mouse Model

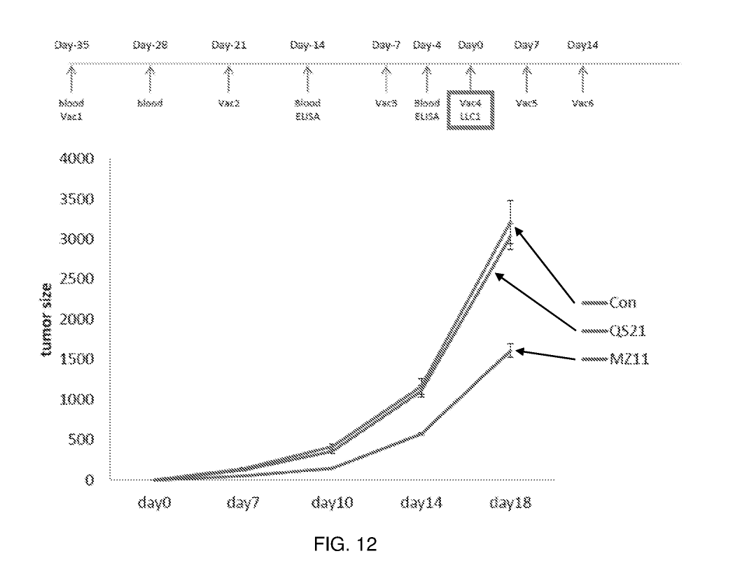

Mice were divided into 3 groups and subcutaneously (s.c.) administered with 1×PBS (control), 20 μg of QS-21 alone or 6 ug of MZ-11-Globo H glycopeptide plus 20 μg of QS-21 at a 2-week interval. Seven days after third vaccination, mice were s.c. implanted 1×10⁵ of LLC1 cells and were concomitantly vaccinated again. The vaccination interval was changed to 7 days after tumor innoculation. Tumor size was measured by caliper at day 7, 10, 14 and 18 after tumor implantation and calculated at length×width×height. FIG. 12 shows that mice treated with the MZ-11-Globo H glycopeptide vaccine demonstrated slower tumor growth (LLC1 cells subcutaneous tumor model in immuno-competent mice).

### Example 10

### Adoptive Transfer of Immunized Serum to Intra-Peritoneal Ovarian Tumor Model Showed Obvious Anti-Tumor Efficacy

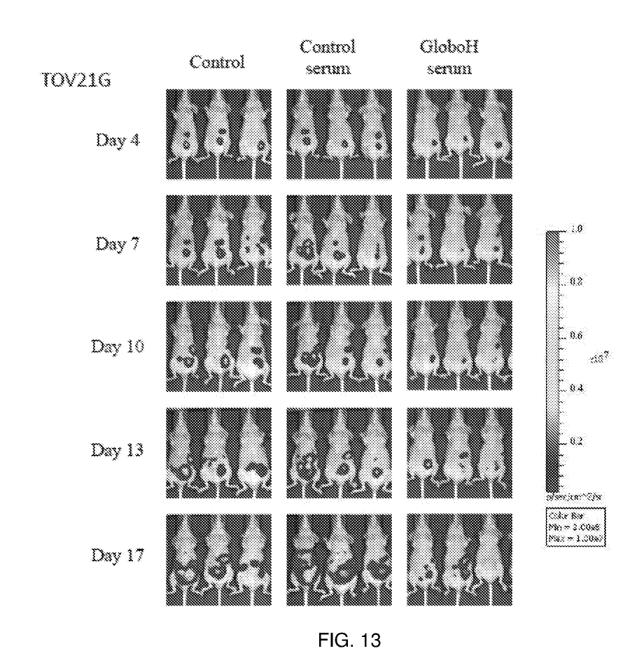

Mice were divided into 2 groups. Serum was collected from group 1 mice without immunization as control. Serum was also collected from group 2 mice vaccinated with MZ-11-Globo H glycopeptide compound three times at a 2-week interval as anti-Globo H serum. One million TOV21G cells were intra-peritoneal (i.p.) implanted into 5-week-old female NU/NU mice (BioLASCO Taiwan). After 4 days, mice were administered with 200 μL of control serum or anti-GloboH serum 3 times a week through i.p. route. Untreated mice were set as control. For monitoring tumor growth, tumor bearing mice were i.p. injected 2004, of luciferin (3.9 mg/ml). The chemoluminescent intensity of each mouse was detected by a non-invasive IVIS system (Xenogen) with fixed exposure condition per batch of experiment. FIG. 13 shows that mice treated by adoptive transfer of serum from mice immunized by MZ-11-Globo H glycopeptide vaccine showed small tumor burden (human ovarian cancer TOV21G cells intra-peritoneal tumor model in immuno-compromised mice).

### Example 11

### Polyvalent Vaccine Efficiently Induces High-Titer Anti-Carbohydrate IgG Antibodies Against Each of Respective Carbohydrate Antigen

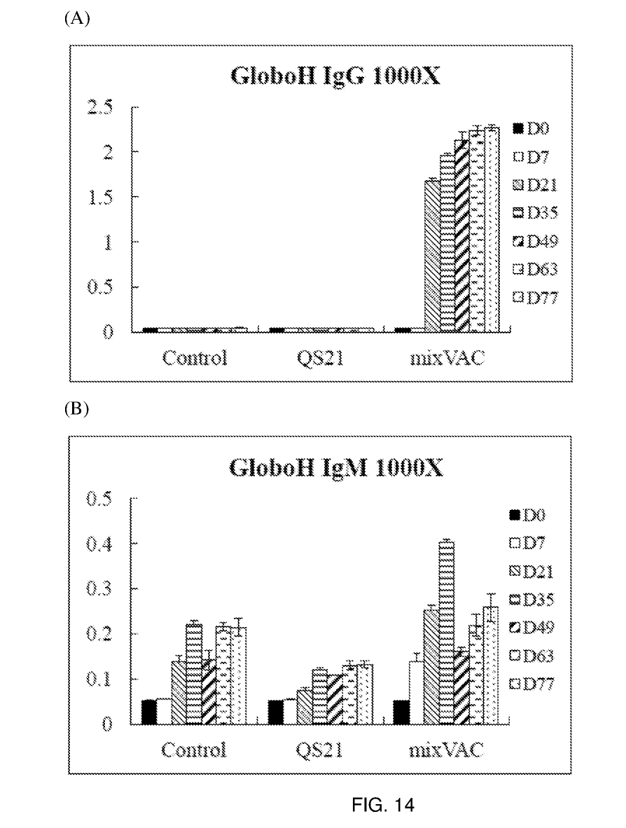

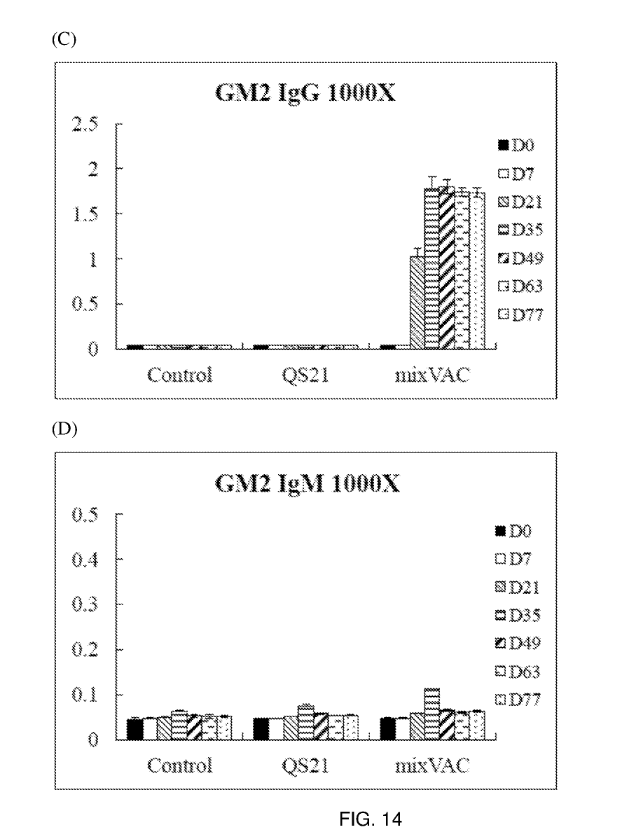

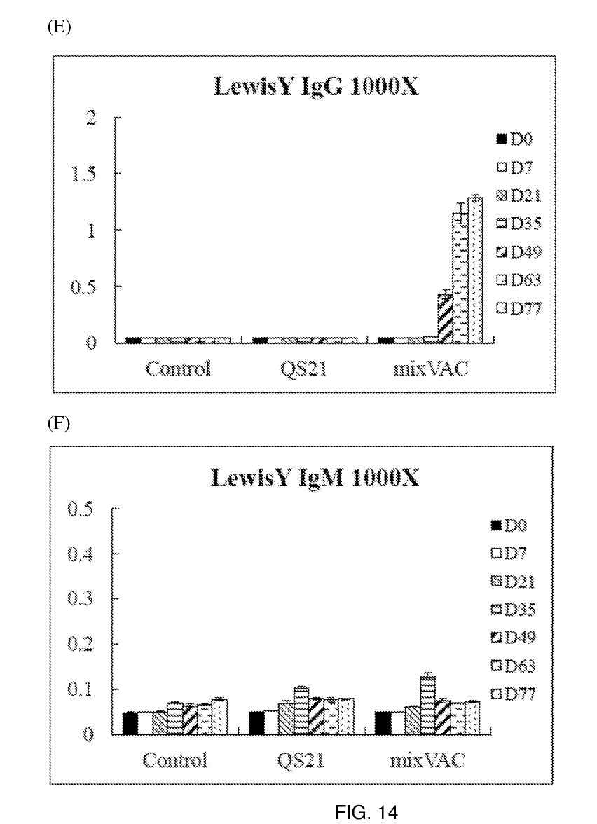

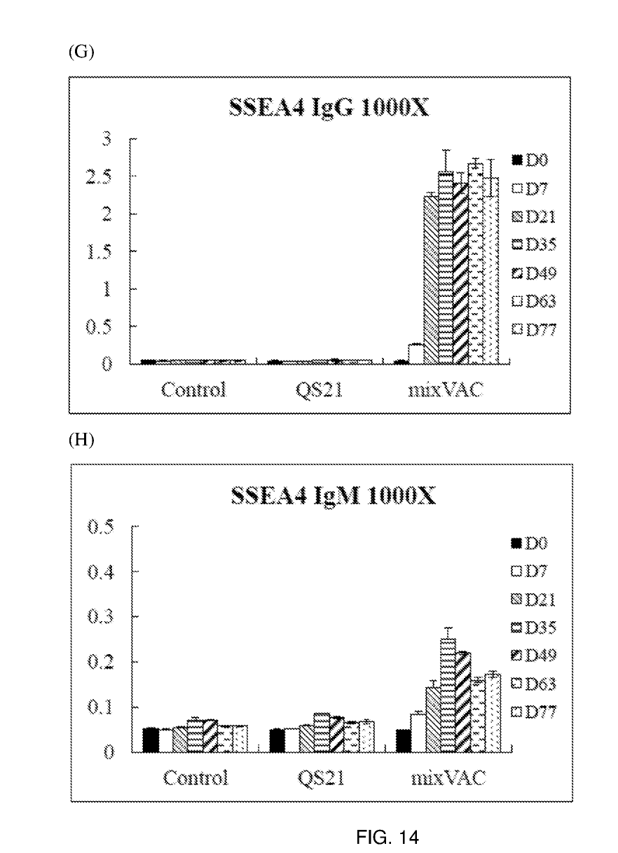

C57BL/6 mice were immunized 6 times with adjuvant (QS-21) alone or admixture of 2 μg MZ-11-Globo H glycopeptide (as prepared in Example 1), or 4 μg SSEA4-PADRE (i.e., glycopeptide compound of structural formula (I), wherein carbohydrate antigen is SSEA4 and pan-DR epitope is sequence of SEQ ID NO: 1) and 2 μg GM2-PADRE (i.e., glycopeptide compound of structural formula (I), wherein carbohydrate antigen is GM2 and pan-DR epitope is sequence of SEQ ID NO: 1) and 4 μg Lewis Y-PADRE (i.e., glycopeptide compound of structural formula (I), wherein carbohydrate antigen is Lewis Y and pan-DR epitope is sequence of SEQ ID NO: 1) plus adjuvant QS21 at a 2-week interval. Anti-sera were harvested at first day and every 7 days after immunization. Control sera were collected from mice without immunization. For ELISA assay, a 96-well Costar assay plate (9018, Corning) were coated with 1 μg streptavidin (21135, Thermo) in 1×PBS overnight at 4° C. and blocked with 1% BSA (ALB001.100, BioShop) in 1×PBS. Then 0.1 μg biotin-conjugated carbohydrate as antigen were loaded and incubated with 1:1000 and 1:10000 diluted serum in the blocking solution, followed by washing in 1×PBS 0.05% Tween 20. Mouse IgG and IgM were detected using HRP-conjugated goat anti-mouse IgG-Fc (1:5000 115-035-071, Jackson Immunoresearch) and HRP-conjugated goat anti-mouse IgM μ chain (1:5000; AP128P, MILLPORE). Color development was performed by adding 100 μL of NeA-Blue solution (010116-1, Clinical Science Products) and stopped with 504, of 2N sulfuric acid. The O.D. value was read at 450 nm subtracted 540 nm as reference. FIG. 14 shows polyvalent vaccines composed of MZ-11-Globo H glycopeptide, GM2-PADRE, Lewis Y-PADRE conjugation mixtures or SSEA4-PADRE, GM2-PADRE, Lewis Y-PADRE conjugation mixtures can induce high-titer of IgG against each of respective carbohydrate antigen (FIG. 14(A): Globo H IgG 1000X; FIG. 14(B): Globo H IgM 1000×; FIG. 14(C): GM2 IgG 1000×; FIG. 14(D): GM2 IgM 1000×; FIG. 14(E): LewisY IgG 1000×; FIG. 14(F): LewisY IgM 1000×; FIG. 14(G): SSEA4 IgG 1000×; FIG. 14(H): SSEA4 IgM 1000×).

What is claimed is:

1. An immunogenic glycopeptide compound, wherein the compound has structural formula (I): 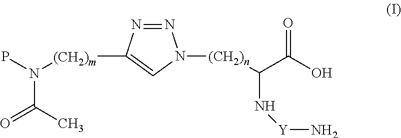
wherein
P is a carbohydrate antigen selected from Globo H, SSEA4, GD2, GD3, GM2, fucosyl GM1, LewisY Le(y), sialyl-Le(x), sialyl-Le(a), TF, Tn and sialyl-Tn;
m=1 to 4;
Y is a pan-DR epitope comprising an amino acid sequence at least 80% identical to AKXVAAWTLKAAA (SEQ ID NO: 1), wherein X is an amino acid residue selected from cyclohexylalanine, phenylalanine, and tyrosine; and
n=1 to 5.

2. The compound of claim 1, wherein m=1.

3. The compound of any one of claims 1 to 2, wherein n=4.

4. The compound of any one of claims 1 to 3, wherein the pan-DR epitope Y consists of the amino acid sequence AKXVAAWTLKAAA (SEQ ID NO: 1).

5. The compound of any one of claims 1 to 3, wherein pan-DR epitope Y consists of the amino acid sequence AKXVAAWTLKAA (SEQ ID NO: 2).

6. The compound of any one of claims 1 to 5, wherein X is cyclohexylalanine.

7. The compound of any one of claims 1 to 6, wherein the carbohydrate antigen P is Globo H.

8. The compound of claim 1, wherein the compound has structure formula (II) 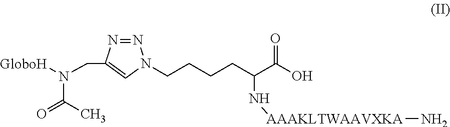
wherein,
GloboH is the carbohydrate antigen Globo H;
X is cyclohexylalanine.

9. The compound of claim 1, wherein the compound has structural formula (III) 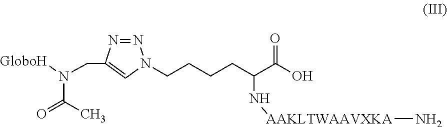
wherein,
GloboH is the carbohydrate antigen Globo H;
X is cyclohexylalanine.

10. A pharmaceutical composition comprising a therapeutically effective amount of an immunogenic glycopeptide compound of any one of claims 1 to 9, and a pharmaceutically acceptable carrier or adjuvant.

11. The pharmaceutical composition of claim 10, wherein the adjuvant is QS21 or aluminum hydroxide.

12. The pharmaceutical composition of any one of claims 10 to 11, wherein the composition is a vaccine.

13. The pharmaceutical composition of claim 12, wherein the vaccine is a polyvalent vaccine comprising two or more immunogenic glycopeptide compounds as defined in claim 1, each of the of the two or more compounds having a different carbohydrate antigen selected from Globo H, SSEA4, GD2, GD3, GM2, fucosyl GM1, LewisY Le(y), sialyl-Le(x), sialyl-Le(a), TF, Tn and sialyl-Tn.

14. The pharmaceutical composition of claim 13, wherein the two or more compounds comprise the carbohydrate antigens: Globo H, SSEA4, GD2, GD3, GM2, fucosyl GM1, LewisY Le(y), sialyl-Le(x), sialyl-Le(a), TF, Tn and sialyl-Tn.

15. A method for preventing and/or treating a cancer in a subject comprising administering to the subject an effective amount of an immunogenic glycopeptide compound of any one of claims 1 to 9.

16. A method for preventing and/or treating a cancer in a subject comprising administering to the subject an effective amount of the pharmaceutical composition of any one of claims 10 to 14.

17. The method of any one of claims 15 to 16, wherein the cancer is a tumor-associated carbohydrate-expressing cancer.

18. The method of any one of claims 15 to 17, wherein the cancer is breast cancer, ovarian cancer, pancreatic cancer, prostate cancer, colorectal cancer or lung cancer.

19. The compound or composition of any one of claims 1 to 14, wherein mice immunized with the compound or composition produce a higher titer of IgG relative to IgM antibodies specific to the carbohydrate antigen.

20. The compound or composition of claim 19, wherein the titer of IgG relative to IgM antibodies specific to the carbohydrate antigen is increased at least about 2-fold, about 4-fold, about 5-fold, or about 10-fold.


SEQUENCE LISTING (2 sequences)

SEQ ID NO: 1 - protein, 13 aa, Artificial Sequence
  Description of Artificial Sequence Synthetic peptide
  MISC_FEATURE (3)..(3): Cyclohexylalanine, Phenylalanine, or Tyrosine
AKXVAAWTLKAAA

SEQ ID NO: 2 - protein, 12 aa, Artificial Sequence
  Description of Artificial Sequence Synthetic peptide
  MISC_FEATURE (3)..(3): Cyclohexylalanine, Phenylalanine, or Tyrosine
AKXVAAWTLKAA